(19) United States
(12) Patent Application Publication
(10) Pub. No.: US 2022/0078986 A1
(43) Pub. Date: Mar. 17, 2022

(54) PRODUCTION OF HYBRID SEEDS LOT USING NATURAL POLLINATION

(71) Applicant: EQUI-nom Ltd., Givat Brenner (IL)

(72) Inventors: Gil SHALEV, Ramot Mehir (IL); Oron GAR, M.P. Lachish Darom (IL); Itay ZEMACH, Rehovot (IL); Ziv ROTENBERG, Rehovot (IL); Michael INLENDER, D.NGalil Elion (IL)

(73) Assignee: EQUI-nom Ltd., Givat Brenner (IL)

(21) Appl. No.: 17/054,264

(22) PCT Filed: May 7, 2019
(86) PCT No.: PCT/IL2019/050516

§ 371 Date: Nov. 10, 2020

Related U.S. Application Data
(60) Provisional application No. 62/669,398, filed on May 10, 2018.

(51) Int. Cl. A01H 1/02; A01H 1/04

(52) U.S. Cl. CPC A01H 1/02; A01H 1/04

(57) ABSTRACT

Methods and hybrid seed are provided which involve growing two varieties of a field crop which is at least partially cross-pollinated in the field, wherein the two varieties are fertile with respect to both male and female functions, are selected to yield specified hybrid(s), and are distinguishable with respect to seed characteristic(s). Collected seeds from the grown field crop are separated into fractions with respect to the seed characteristic, wherein at least one fraction is of hybrid seeds of the specified hybrid(s). For example, the parent varieties may be selected to yield heterotic hybrid(s) which are distinct and separable from the parent non-hybrid seeds with respect to the at least one seed characteristic, e.g., have larger or smaller seeds. Possibly, different traits may be used to separate the hybrid seeds from the two parent variety seeds, such as size, weight or optical parameters that enable sorting out the hybrids from the overall crop.

### CROSS-REFERENCE TO RELATED APPLICATIONS

This application is a National Phase Application of PCT International Application No. PCT/IL2019/050516, International Filing Date May 7, 2019, claiming priority of United States Provisional Patent Application No. 62/669,398, filed on May 10, 2018, which is hereby incorporated by reference.

### BACKGROUND OF THE INVENTION

### 1. Technical Field

The present invention relates to the field of hybrid production methods in plants, and more particularly, to hybrid production using natural pollination.

### 2. Discussion of Related Art

The terms “heterosis” or “hybrid vigor” describes the genetic phenomenon in which a hybrid exhibits an increased function of one or more biological qualities or traits, beyond the respective quality in its parents (in either direction of the trait, such as larger or smaller size). Heterosis has been long utilized in plant breeding as it provides several agronomic advantages for the offspring over the parental lines such as higher yield and environmental stability.

A seed is an embryonic plant enclosed in a protective coating. It is the product of the ripened ovule which develops after fertilization. The seed consists of, from the outer to the inner parts, the testa (coating), the pericarp, the seed storage organ and the embryo. In endospermic seeds the endosperm is the major storage organ while in non-endospermic seeds such as legumes (pea, cowpea, broad bean and others) the cotyledons accumulate the seed reserves. There is a third group of perispermic seeds like sugar beet (Beta vulgaris), in which the perisperm is derived from the nucellus and stores the seed reserves. These types of storage organs are described in Gallardo et al. 2008, Reserve accumulation in legume seeds, Comptes Rendus Biologies 331(10): 755-762.

Pollination is the process by which pollen is transferred from the anther (male part) to the stigma (female part) of the plant, thereby enabling fertilization and reproduction. The pollen grain (gametophyte) containing the male gametes are naturally transported to the stigma by biotic vectors (like insects, animals) and/or by abiotic vectors (like wind, water), where it germinates and its pollen tube grows down the style to the ovary. The two gametes in the pollen grain travel down the tube to where the gametophyte(s) containing the female gametes are held within the carpel. One nucleus fuses with the polar bodies to produce the endosperm tissues, and the other with the ovule to produce the embryo and the cotyledons.

Pollination can be accomplished by cross-pollination, also called allogamy, which occurs only when pollen is delivered to a flower from a different plant, or by self-pollination, also called autogamy or geitonogamy, which occurs when pollen from one flower pollinates the same flower or other flowers of the same individual, respectively.

Unlike the embryo, which contain two nuclei (the maternal nucleus and the paternal nucleus), the endosperm is formed by multiple fertilizations, which usually result in 3N endosperm with two nuclei from the maternal parent and only one from the paternal parent. Although the endosperm is not always triploid, it always contains more maternal genes than paternal genes. Maternal and paternal effects in seeds originate via the endosperm. As a consequence of the differential dosage of male and female genes on the endosperm, differences in seed characteristics are occurring in size, shape, color, weight, enzymes contents, proteins contents, nutrition contents and metabolite contents. The female parent may have a more important role in determining seed characteristics, as reviewed in D. A. Roach and R. D. Wulff 1987, Maternal effects in plants, Annual review of ecology and systematics 18:209-235.

Polyploidy is defined as the existence of more than two sets of complete genomes all over the plant cells. Polyploidy in crop plants is a known phenomenon and may affect the vegetative organ size such as the size of potato tubers and also the fruits like in strawberries, as described in H. Weiss-Schneeweiss et al. 2013 (Evolutionary Consequences, Constraints and Potential of Polyploidy in Plants Cytogenet Genome Res 140:137-150). Fertilization in polyploids is possible when the parents comprise even sets of genomes, as for example in the hybridization of tetraploid crop plants with their diploid relatives. When hybridization occur between relatives which comprise uneven set(s) of genomes, fertilization leads to seed abortion and irregular development of hybrid seeds. Hybridization in polyploid crops can contribute to hybrid vigor in specific trait(s) due to greater level of heterozygosity. Examples for such traits are fruit size in apple (Malus domestica), petal size in rose (Rosa hybrida) and the size and weight of the hybrid seeds. In other cases, traits vigor may be reduced resulting in the loss of some trait performance such as fertility (e.g., Ranney 2006, Polyploidy: From Evolution to New Plant Development. Combined Proceedings International Plant Propagators' Society, Volume 56, 137-142).

Seed size and or seed weight play an important role in plants fitness and therefore contribute to plant evolution. Seed size is a common subject in many breeding programs since the edible part of the crop is the grain such as in wheat, barley, corn, sesame and more. Moreover, plant vigor is found to be con⁻elated to its seed size and weight. Due to its complicated manner, many genes control this trait. Seed size is also affected by the ploidy level of the plant as shown in Miller et al., 2012 (Ploidy and hybridity effects on growth vigor and gene expression in Arabidopsis thaliana hybrids and their parents. G3 2,505-513). Still, to date there is no evidence of heterotic effect in the F1 (filial 1, first generation hybrids) seed/embryo size nor evidence of heterotic effect in the F1 seed weight in crop plants (the latest publication relating to this subject which was found by the inventors is Ashby, E. 1930, Studies in the Inheritance of Physiological Characters: I. A Physiological Investigation of the Nature of Hybrid Vigour in Maize, Annals of Botany, 44:2, 457-467).

The genetic regulation of seed size is a complicated matter because one paternal genome contribution and at least two maternal genome contributions determine the endosperm genetics, whereas the genomic contribution to the embryo and cotyledons is equal from both parents. The relative role of maternal and paternal control of seed characteristics might therefore be expected to be largely determined by the relative mass contributed by the three generations represented in the seed, nevertheless, the food supply for the whole unit is entirely maternal. To what degree seed size is determined by the forces of supply from the parent and by the forces of demand from the developing embryo and endosperm remain disputable with the current knowledge, as overviewed in Linkies et al. 2010 (The evolution of seeds, New Phytologist 186:817-831).

Current hybrid production methods utilize different natural and artificial barriers to prevent self-pollination. For example, such barriers include dioecious flower anatomy, self-incompatibility, manual emasculation and genetic or induced male sterility. Without these barriers the harvested seed lot contains different proportions of four types of seeds: self and cross-pollinated seeds from parent 1 plants and self and cross-pollinated seeds from parent 2 plants. Furthermore, it is not an uncommon experience to realize that the seed lot cannot be labeled nor marketed as hybrid seeds lot without using such barriers.

### SUMMARY OF THE INVENTION

The following is a simplified summary providing an initial understanding of the invention. The summary does not necessarily identify key elements nor limit the scope of the invention, but merely serves as an introduction to the following description.

One aspect of the present invention provides a method of obtaining specified hybrid seeds, the method comprising: growing two varieties of a field crop, wherein the two varieties: the varieties are at least partially cross-pollinated in the field, are fertile with respect to both male and female functions, are selected to yield at least one specified hybrid, and are distinguishable with respect to at least one seed characteristic; collecting seeds from the grown field crop, and separating, from the collected seeds, at least one fraction of hybrid seeds of the at least one specified hybrid, wherein the at least one separated fraction is distinct and separable, with respect to the at least one seed characteristic, from seed fractions of the non-hybridized two varieties.

Another aspect of the present invention provides a method of producing heterotic hybrid seeds which are different from the respective parent varieties by at least one seed characteristic, e.g., size, e.g., heterotic hybrid seeds may be larger than the seeds of their parent plants. The plant varieties are at least partially cross-pollinated in the field, are fertile with respect to both male and female functions, are selected to yield at least one specified hybrid, and are distinguishable with respect to at least one seed characteristic; and the method may comprise collecting seeds from the grown field crop, and separating, from the collected seeds, at least one fraction of hybrid seeds of the at least one specified hybrid, wherein the at least one separated fraction is heterotic and separable, with respect to the at least one seed characteristic, from seed fractions of the non-hybridized two varieties.

These, additional, and/or other aspects and/or advantages of the present invention are set forth in the detailed description that follows; possibly inferable from the detailed description; and/or learnable by practice of the present invention.

### BRIEF DESCRIPTION OF THE DRAWINGS

For a better understanding of embodiments of the invention and to show how the same may be carried into effect, reference will now be made, purely by way of example, to the accompanying drawings in which like numerals designate corresponding elements or sections throughout.

In the accompanying drawings:

FIGS. 1A and 1B are high-level conceptual illustrations of distributions of a seed characteristic, according to some embodiments of the invention.

FIGS. 2 and 3 are high-level schematic flowcharts illustrating a method, according to some embodiments of the invention.

FIGS. 4 and 5 illustrate examples for seed distributions with respect to the seed characteristic seed weight, in peas and cowpeas, respectively, according to some embodiments of the invention.

FIGS. 6A and 6B provide examples for separation of hybrid seeds of cowpeas and peas, respectively, using two different seed characteristics, such as seed color providing separation between the first (female—pollen receiving) parent and seed size providing separation between the second (male—pollen donating) parent, according to some embodiments of the invention.

FIG. 7 provides examples for separation of hybrid seeds of sesame from the respective parent seeds using near infrared (NIR) spectroscopy, according to some embodiments of the invention.

### DETAILED DESCRIPTION OF THE INVENTION

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before at least one embodiment of the invention is explained in detail, it is to be understood that the invention is not limited in its application to the details of construction and the arrangement of the components set forth in the following description or illustrated in the drawings. The invention is applicable to other embodiments or of being practiced or carried out in various ways. In addition, it is to be understood that the phraseology and terminology employed herein is for the purpose of description and should not be regarded as limiting.

Embodiments of the present invention relate to using heterotic characteristics of the hybrid seeds in comparison to the seeds of their male and female parent varieties. Non-limiting examples for heterotic characteristics comprise seed size, seed weight, seed color, seed shape, absorption and/or reflection spectra (e.g., in NIR) and more. Certain embodiments comprise separating heterotic hybrid seeds, which are distinct and separable from both parent varieties due to at least one heterotic trait in which they diverge from one or both parents, e.g., larger or smaller seeds. It is noted that the heterotic trait is manifested in the hybrid F1 seeds, and not necessarily, or preferably not, in F2 (filial 2, second generation hybrid) seeds produced from the heterotic hybrid seeds. It is emphasized that heterosis has not been used as breeding feature in F1 hybrid seeds, which are not themselves the aimed product of the breeder.

Provided are a method of production of hybrid seeds lot using natural pollination, as well as corresponding systems and devices involved in the production and separation, related sowing patterns, selection of parents and resulting seed assemblies with given distribution characteristics. Hybrid seeds are produced using two parents, which are intermingled, in the hybrid seed production field. Applying the developed algorithm enables to exploit the genetic differences in seed characteristics (such as shape, size, color, weight and internal components) between the parents by employing post-harvest techniques to increase the percentage of hybridity. Field trial evaluation for the seeds lot provide verification of the required level of hybridity which allowed its labeling and marketing as hybrid seeds lot.

In certain embodiments, hybrid seeds of two varieties of a field crop which are at least partially bi-directionally cross-pollinated in the field are provided, wherein the two varieties are fertile with respect to both male and female functions are selected to yield at least one specified hybrid, and are distinguishable with respect to at least one seed characteristic. In collected seeds of the field crop, at least one fraction of hybrid seeds of the at least one specified hybrid is distinct and separable, with respect to the at least one seed characteristic, from seed fractions of the non-hybridized two varieties.

Certain embodiments overcome the challenge of producing hybrid seeds using natural pollination by exploiting or breeding differences in seed characteristics. Genetic differences between the parents in the seed's endosperm, perisperm and embryo characteristics, such as size, shape, color and metabolic content, are affected by the genetic differences between the parental lines and the epigenetic interaction of the created seed, resulting in different characteristics in the offspring seeds, as illustrated schematically in Tables 1A and 1B.

As is schematically shown in Tables 1A and 1B, different combinations of parent plant types results in seeds having different genomic constitution in the endosperm and the embryo, cotyledons and/or perisperm seed tissues, which result in variety in the offspring seeds. Specifically, the four types of seeds would differ in certain characteristics relating to seed weight or size. It is noted in endospermic seeds that not only do hybrids differ from the varieties in their genomic composition, but the hybrids also differ among themselves in their genomic composition, as they have namely different endosperm doses of the parent genomes. On the other hand, in non-endospermic seeds, hybrid 1 and hybrid 2 are completely similar in their genomic composition. Table 1B shows the seed genetic characteristic in case of tetraploid seed hybridization and also the possible hybrid coming from hybridization of tetraploid seeds with diploid seeds.

It is further noted that the ploidy level of cotyledons is 2N, as it originates from the fertilization between maternal and paternal gametes. Therefore, the genetic basis of non-endospermic seeds is different from the genetic basis of endospermic seeds and the role of the endosperm in these kinds of seeds is peripheral.

Certain embodiments comprise a process of developing hybrid seeds based on designing (i.e., selecting or breeding) parental lines with specific genetic variation that increases phenotypic differences in seed characteristics between the two parental lines, to create traceable differences in seed characteristics between the hybrid seeds and the non-hybrid seeds and thus to enable differentiation of the four different seed lots. Certain methods combine breeding the parental lines for specific seed characteristics and seed production uses natural pollination.

Methods and hybrid seeds are provided which involve growing two varieties of a field crop which is at least partially cross-pollinated in the field, wherein the two varieties are fertile with respect to both male and female functions, are selected to yield specified hybrid(s), and are distinguishable with respect to seed characteristic(s). Collected seeds from the grown field crop are separated into fractions with respect to the seed characteristics, wherein at least one fraction is of hybrid seeds of the specified hybrid(s). For example, the parent varieties may be selected to yield heterotic hybrid(s) which are distinct and separable from the parent non-hybrid seeds with respect to the at least one seed characteristic, e.g., have larger or smaller seeds. Possibly, different traits may be used to separate the hybrid seeds from the two parent variety seeds, such as size, color, shape, weight or optical parameters, such as near infra red (NIR) spectral signature that enable sorting out the hybrids from the overall crop.

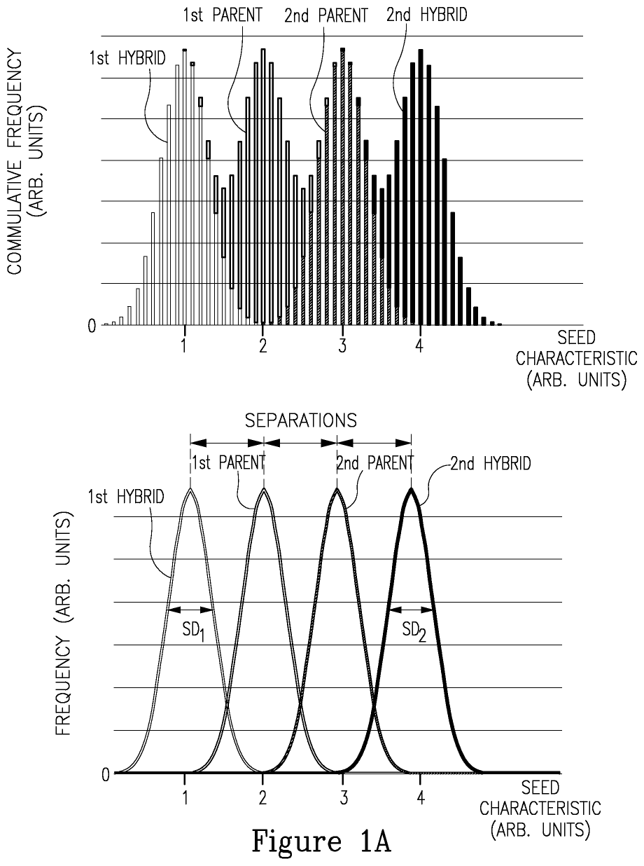

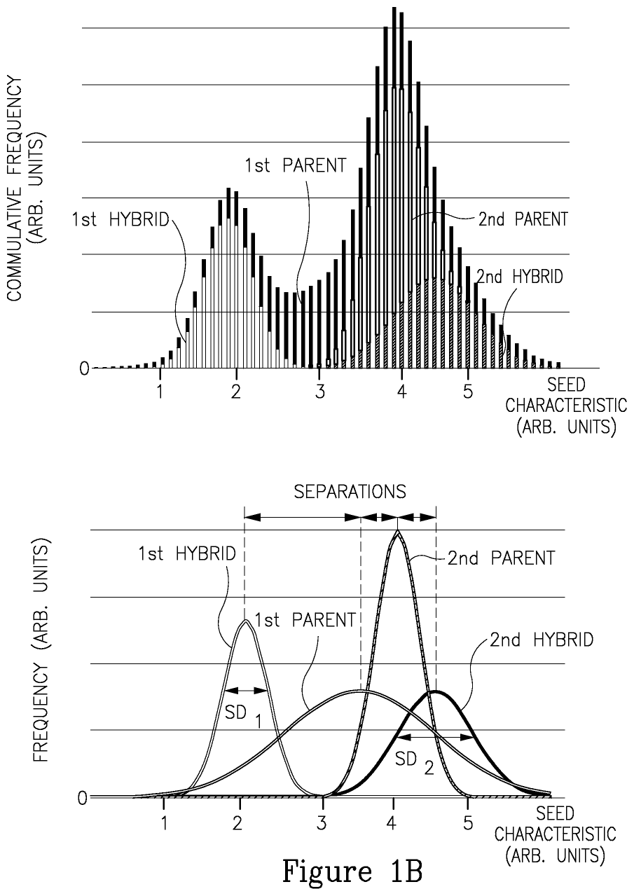

FIGS. 1A and 1B are high-level conceptual illustrations of distributions of a seed characteristic, according to some embodiments of the invention. The illustrations depict two exemplary distributions which may characterize given parent varieties or may be set as breeding targets. The x-axis denotes an abstract seed characteristic (which may represent e.g., a seed size, a seed weight, a visual or an optical characteristic of the seed etc., the units are arbitrary) and the y-axis denotes a frequency of the value of the seed characteristic (in abstract terms). The bottom diagrams present the distributions of the parents and the hybrids in an overlapping manner, and the top diagrams present the distributions of the parents and the hybrids in a cumulative manner, e.g., the former depicts breeding targets and the latter depicts resulting seed distributions from the produced crops. It is noted that while FIGS. 1A and 1B depict schematically Gaussian distributions, the invention is in no way limited to Gaussian distributions and may be likewise applicable to any theoretical or empirical distribution of seed characteristics, using equivalent calculation of mean values and standard deviations (e.g., with the mean values and the standard deviations being statistical measures of empirical distributions of the respective seeds).

FIG. 1A presents a case of distinct distributions of a seed characteristic, which have little overlap and enable separation of the hybrids according to the seed characteristic with a high purity level. For example, in FIG. 1A, the large majority of seeds are within the range of ca. 1±0.3 is of the first hybrid and the large majority of seeds within the range of ca. 4±0.3 is of the second hybrid.

FIG. 1B presents a case of less distinct distributions of the seed characteristics, which have significant overlaps yet nevertheless enable separation of the hybrids according to the seed characteristics. For example, in FIG. 1B, a majority of seeds within the range of ca. 1-2.5 are of the first hybrid and a majority of seeds within the range of ca. 4-6 are of the second hybrid. It is noted that changing the range of the seed characteristic(s) from which the seeds are taken provides control over the purity of the collected seeds.

In general, in each application of the disclosed methods to a specific crop, both with respect to breeding the parent varieties and with respect to selecting the actual parents, considerations such as the separations among the distributions of the parents and the hybrids, as well as the degree of purity that is achievable by selecting certain portions of the overall distribution, e.g., the portion selected from the overall crop that is collected in the field (including a mix of parent and hybrid seeds) may be made anew to accommodate the disclosed method to the specific crop at hand. For example, depending on the technical separation capabilities, parent varieties may be selected to have their distribution means similar or apart, while being distinct from the means of the hybrids, or at least have much smaller standard deviations, to allow separation. Hybrids may be selected to be separable from the adjacent parent by having their distribution means apart by 0.2, 0.5, 1 etc. times the sum of the respective standard deviations. It is noted that as crops are usually bred toward a similar value of additive seed characteristics such as seed size and seed weight in both parent crop varieties, hybrid seeds with heterotic (larger or smaller) seed characteristics may be separable from non-hybridized seeds even when parent seed characteristics are close or identical. It is emphasized that while common breeding practices aim at uniform seed characteristics, the present invention surprisingly selects or breeds the parent varieties to yield heterotic F1 hybrid seeds which are different in at least one seed characteristic from the parents and is hence separable.

These aspects are indicated schematically in FIGS. 1A, 1B by the arrows marking the separations between the respective distribution mean values and the corresponding standard variation (SD, indicated only for the parent varieties); and by the arrow marking the selection and respective purity of one type of seeds in the selection—SEL₁ and SEL₂ indicate possible selected portions of hybrid seeds 1 and 2 respectively. It is noted that if a higher level of purity is required, parent varieties may be selected and/or bred to provide narrower parent distributions and/or parent distributions which are more removed from the hybrid distributions. Alternatively, or complementarily, a smaller portion of seeds (from the corresponding location in the cumulative distribution) may be selected. In case a lower level of purity is required the criteria outlined above (e.g., width of parent distributions, separation of parent distribution for hybrid distribution and separation criteria) may be relaxed.

In practice, parent varieties may be selected to exhibit distributions which allow hybrid seed separation by the seed characteristic(s), such as seed size, weight or color. It is noted that such selection is challenging and unexpected, as normally parent varieties are selected to have similar distributions of seed characteristics in order to yield uniform hybrids. In certain embodiments, parent varieties may be bred to yield hybrid seeds with required properties as well as to have differing distributions with respect to one or more seed characteristics which are intended to be used for separating the hybrid seed.

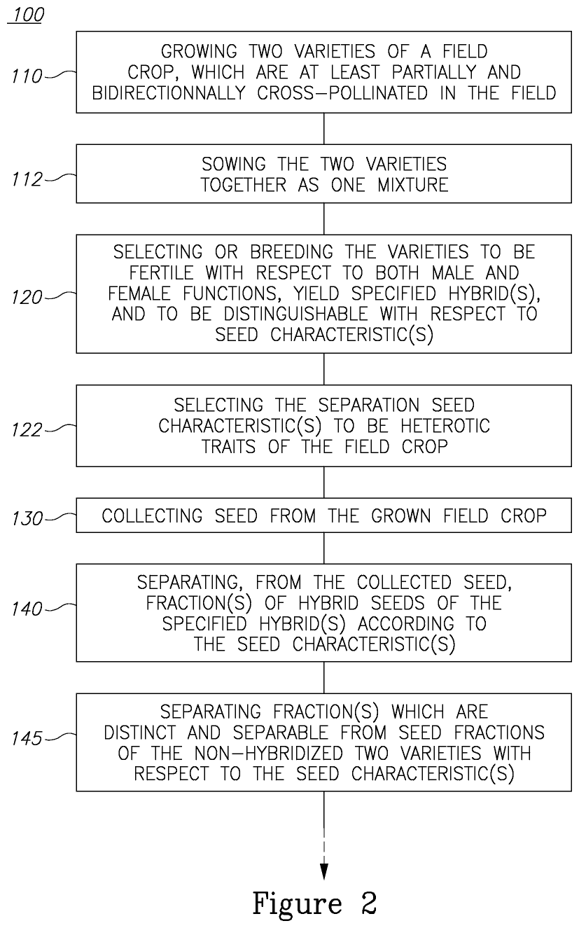

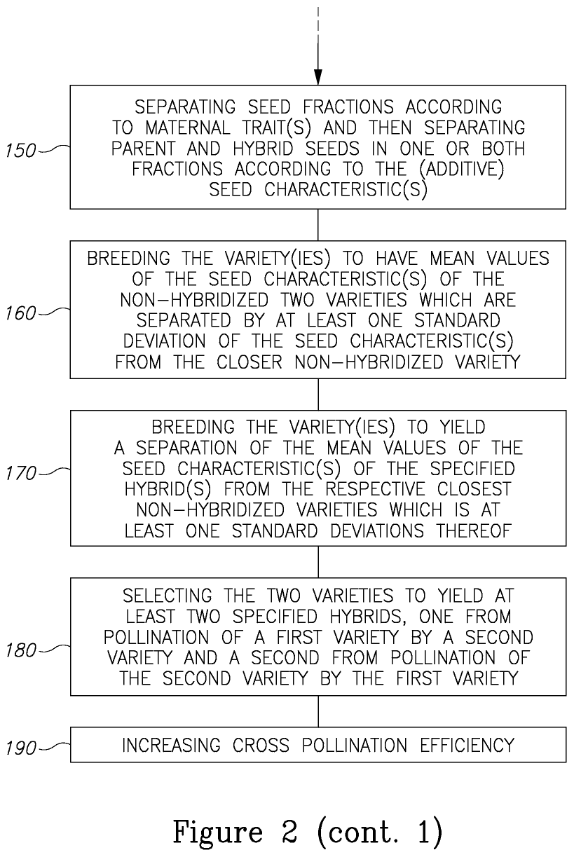

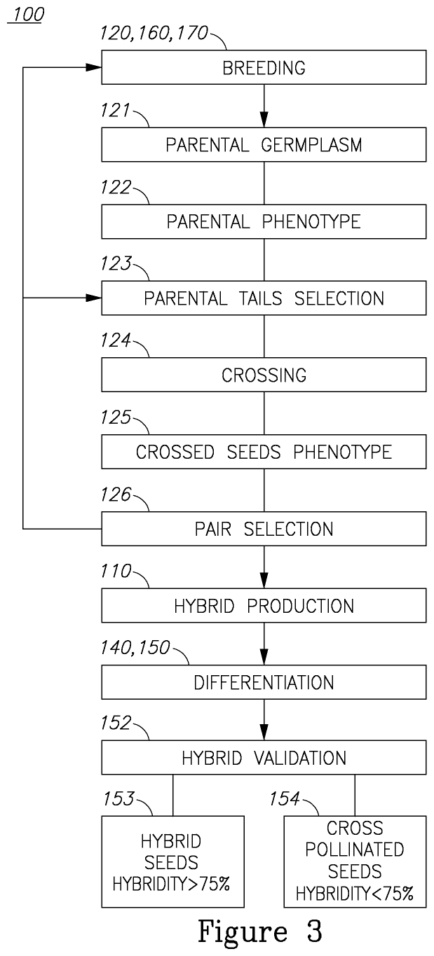

FIGS. 2 and 3 are high-level schematic flowcharts illustrating a method 100, according to some embodiments of the invention. Method 100 may comprise growing two varieties of a field crop which is at least partially cross-pollinated in the field (stage 110), e.g., the varieties are at least partially and bidirectionally cross pollinated in the field, e.g., by sowing the two varieties together as one mixture (stage 112), after selecting or breeding the varieties to be fertile with respect to both male and female functions, yield specified hybrid(s), and to be distinguishable with respect to seed characteristic(s) (stage 120). Method 100 may further comprise selecting the separation seed characteristic(s) to be heterotic traits of the field crop (stage 128).

Method 100 may further comprise collecting seeds from the grown field crop (stage 130) and separating, from the collected seeds, fraction(s) of hybrid seeds of the specified hybrid(s) according to the seed characteristic(s) (stage 140), for example, separating fraction(s) of at least one hybrid, which are distinct and separable from seed fractions of the non-hybridized two varieties with respect to the seed characteristic(s) (stage 145).

Separation 140 and/or 145 may be carried out by various machines, and according to different seed characteristics. For example, seed shape may be used as a seed characteristic by applying to the harvested seeds a spiral separator, which uses gravity and centripetal force to separate rounder shaped seeds from flatter seeds. In another example, seed size may be used as a seed characteristic by applying to the harvested seeds an indented cylinder, configured to separate larger seeds from smaller seeds. In another example, seed weight may be used as a seed characteristic by applying to the harvested seeds a gravity table, which uses both air and gravity to separate the heavier seeds from the lighter seeds. In another example, internal seed components or compounds may be used as a seed characteristic by applying to the harvested seeds NIR (near infrared) spectroscopy technology or other spectroscopy-based technologies for separating the seeds based on specific signals which are linked to the internal components or compounds. Other seed characteristics as well as combinations of seed characteristics may be used to separate the hybrid seeds from the parent seeds and possibly from each other.

In certain embodiments, separation may be carried out in two stages. A first separation may be carried out between seed fractions (i) of one parent variety and a close hybrid on the one hand and (ii) of the second parent and a close hybrid on the other hand (referring as an example to FIGS. 1A, 1B—the first separation is between 1^{st} parent+1^{st} hybrid on one hand and 2^{nd} parent+2^{nd} hybrid on the other hand). A second separation may be then carried out within each (or at least one) group, e.g., between parent seeds and hybrid seeds in each group (referring as an example to FIGS. 1A, 1B—the second separation is between 1^{st} parent and 1^{st} hybrid in the first group, and between 2^{nd} parent and 2^{nd} hybrid in the second group). Different seed characteristics may be used in the first and second separations, e.g., seed color (as a maternal characteristic) may be used in the first separation while seed size or weight may be used for second separation. Advantageously, using a maternal characteristic may enable efficient separation between hybrids 1 and 2 as they result from different parent combinations (see Tables 1A, 1B and FIG. 8 for an example). Seed color may be used as a seed characteristic by applying to the harvested seeds a color sorter in which an electronic eye adjusted to identify color differences is used to separate seeds having different colors. In certain embodiments, only hybrid 1 or only hybrid 2 may be separated, e.g., if maternal or paternal hybrids are preferred (respectively). In this case sowing proportions of the parent varieties may deviate from 1:1 in order to enhance the fraction of one type of hybrid seed over the other.

In certain embodiments, method 100 may comprise separating seed fractions according to maternal trait(s) and then separating parent and hybrid seeds in one or both fractions according to the (heterotic) seed characteristic(s) (stage 150).

In certain embodiments, method 100 may further comprise breeding (or selecting) the variety(ies) to have mean values of the seed characteristic(s) of the non-hybridized two varieties which are separated by at least a sum of the standard deviations of the seed characteristic(s) of the non-hybridized two varieties (stage 160). Such separation of the distributions of the seed characteristic(s) may ensure the ability to separate the hybrid seed from the (pure) parent seeds efficiently.

In certain embodiments, method 100 may further comprise breeding (or selecting) the variety(ies) to yield a separation of the mean value of the seed characteristic(s) of the specified hybrid(s) from the closest non-hybridized variety which is at least a sum of the standard deviations thereof (stage 170). Such separation of the distributions of the seed characteristic(s) may ensure the ability to separate the hybrid seed from the (pure) parent seeds efficiently.

In certain embodiments, method 100 may further comprise selecting the two varieties to yield at least two specified hybrids, one from pollination of a first variety by a second variety and a second from pollination of the second variety by the first variety (stage 180). By selection and/or breeding of the parent varieties, the mean values of the at least one seed characteristic of the first and/or second seed fractions (of the corresponding first and/or second varieties) may be separated by at least a sum of the standard deviations of the at least one seed characteristic of the respective hybrid and corresponding parent variety. Separation of the hybrid seed from the closest parent variety seeds according to seed characteristic(s) may be enabled by the different donations by the male parent and by the female parent to the seed characteristic(s), as explained above (see, e.g., Tables 1A and 1B). In certain embodiments, pollination characteristics may be manipulated or selected to favor the formation of one hybrid over the other and/or over the adjacent parent seeds, e.g., to simplify separation or increase separation efficiency. For example, wind pollinated crops may be grown with one of the parents upwind (when such conditions prevail in the growing region) and thus influence the relative frequency of the hybrids' and the parents'seeds.

Method 100 may further comprise increasing cross pollination efficiency (stage 190) by any of the following means (see also below). In cases one parent variety is predominantly female and another parent variety is predominantly male (e.g., maternally affected traits), the parent varieties may be sown as a mixture to increase the efficacy of cross-pollination. In such cases, predominantly male varieties may be sown in larger proportion than predominantly female varieties to increase the availability of pollen and thus increase the cross-pollination proportion of the predominantly female variety. Predominantly female variety may be selected or bred to exhibit at least partial male sterility (genetic and/or induced) to increase the proportion of cross pollination seeds from the predominantly female plant (by reducing the number of self-seeds therefrom). Possibly, given sufficient cross-pollination, predominantly male and predominantly female varieties may be spatially separated and separately harvested to simplify separating the hybrid seeds from each harvest. Cross pollination may be enhanced and refined by applying biotic and abiotic factors such as introduction of bees or bee attracting means, using artificial abiotic effects such as wind blowers, plant shakers etc. and locating the fields under consideration of bee behavior and propagation of abiotic pollination agents.

FIG. 3 schematically illustrates breeding steps as part of the realization of method 100, according to some embodiments of the invention. Breeding the parental lines (stage 120, also stages 160, 170) may be carried out to achieve a wide genetic diversity which contains the product definition traits and diversity in seed characteristics such as: shape, size, weight, color and internal seed components (enzymes, nutrition, protein and metabolites). For one or more seed characteristics which are defined in advance as the “differentiation characteristic” (DC), breeding 120 may be conceived to set a wide variation of the DC for the ongoing process, which may be characterized in the parental germplasm 121. Alternatively or complementarily, Parental varieties may be selected to have similar values of one or more heterotic characteristics, enhancing the distinction and separability of the heterotic hybrid seeds from the parent seeds. Parental phenotype 122 may be measured in several repetitions and for each parental line; for example, the mean value and standard deviation (SD) of the respective distributions may be calculated. Parental tails selection 123, referring to the tails of the distribution, may be carried out e.g., for the lines with the lower and upper 10% of the DC values (see example in Table 2 and FIG. 5 below).

Parent selection or breeding 120 may aim at modifying the seed characteristic distribution across the crop to enable effective separation of the required hybrid seed. Crop lines with SD higher than the average SD may be dropped out to achieve increased stability of the trait performance (the trait being different from the seed characteristics, and set as a performance requirement from the hybrid seeds or plants grown therefrom). In case of low diversity in the measured characteristic, breeding 120 may be intensified and conducted to introduce new diversity or even change the DC. For example, manually reciprocal crossing 124 between the selected parental lines may be carried out as a preliminary stage to yield the parental varieties to be grown. Crossing 124 may e.g., comprise recurring crossing after determining crossed seeds phenotypes 125 (with respect to the aims of breeding 120) and selecting specific pairs for further crossing 126. Different traits may be bred in different directions (uniformity or diversity of the parent varieties) to provide required hybrid seed characteristics (e.g., heterosis) and required F2 plant traits.

Method 100 may further comprise evaluation and validation of the hybrid seed lot (stage 152). For example, 1000 seeds may be selected after differentiation phase 140 and/or 150 and sown. The mature plants may be phenotyped and divided into three groups: first parent (e.g., predominantly female plants), second parent (e.g., predominantly male plants) and hybrids. The hybrid seeds lot may then be defined as “hybrid seeds” 153 if the percentage of hybridity is exceeding 75% or as “Cross pollinated seeds” 154 if the percentage of hybridity is below 75%. Clearly, any form of evaluation may be applied to confirm appropriate or required separation levels.

In certain embodiments, the following steps may be taken to implement breeding 120 in method 100. In N Parents, each with X weight measurements, the mean and standard deviation of the seed characteristic are calculated. Some of the N parents are then separated into two groups with respect to the seed characteristic (e.g., big seeds and small seeds). For example, a certain distance from the overall average of the N parents may be selected as a threshold for inclusion in the two groups. Several parents are selected from each group according to seed characteristics and possibly statistical measures (e.g., three biggest from the big seeds group and three smallest from the small seeds group; or extreme parents with small standard deviations with respect to other extreme parents in each group). Hybrids from crossings of the selected parents are then evaluated according to their required traits, seed characteristics, and statistical measures of these parameters to identify hybrids with required traits, and (possibly narrow) distributions of seed characteristics which are distinct from the distributions of the seed characteristics of the parents. Selected hybrids and possibly parents may be used for further breeding 120 until required traits and seed characteristics are reached, the parents of the respective hybrids are then used for growing the hybrid seeds in production.

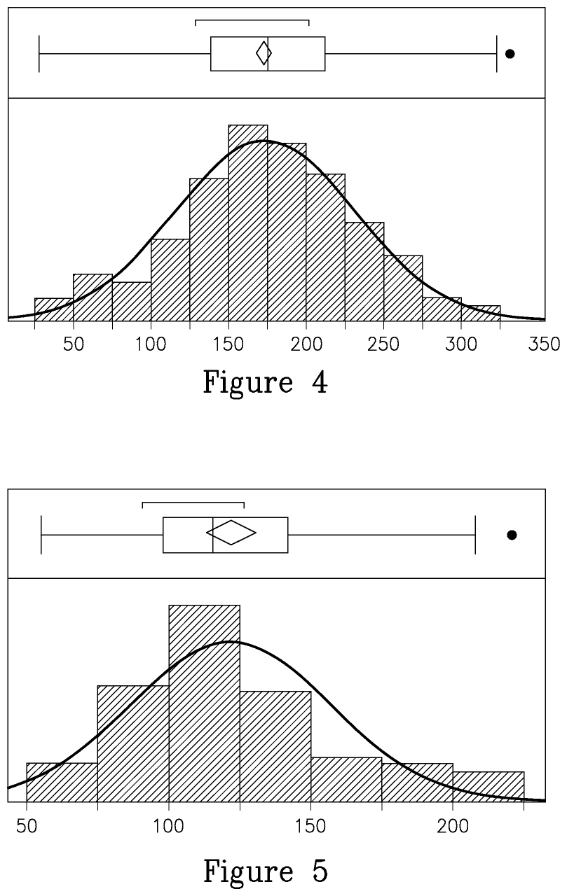

FIGS. 4 and 5 illustrate examples for seed distributions with respect to the seed characteristic seed weight (as 1000 grain weight, TGW, in grams which is equal to the average seeds weight in milligrams), in peas and cowpeas, respectively, with Table 2 providing the distribution of seed weights, according to some embodiments of the invention.

TABLE 2
An exemplary lines distribution with respect to the seed
characteristic seed weight, in peas and in cowpeas.
Mean seed weights (mg) are for respective fractions.

| Quantiles | Cowpea | Pea |
|---|---|---|
| 100% (max) | 220.0 | 330.0 |
| 99.5% | 220.0 | 322.5 |
| 97.5% | 207.6 | 282.9 |
| 90% | 176.0 | 247.8 |
| 75% (3^{rd} qntl) | 142.2 | 212.0 |
| 50% (med) | 115.6 | 175.1 |
| 25% (1^{st} qntl) | 97.8 | 138.0 |
| 10% | 83.0 | 99.3 |
| 2.5% | 58.7 | 53.2 |
| 0.5% | 55.6 | 28.0 |
| 0 (min) | 55.6 | 28.0 |

Tables 3A and 3B exemplify in a non-limiting manner the comparison of different parents as varieties for hybridization, with respect to the resulting distribution parameters, in cotton and in pea, respectively. Tables 3A and 3B illustrate examples that may serve as a basis for a crossing stage (124-126) in the breeding of parental lines to yield a hybrid variety. Six repetitions of each line have been measured to calculate mean value and SD (not shown). The average thousand seed weight (TGW; in grams) is shown for each combination, together with the mean values and standard deviations for each combination.

The data in Tables 3A and 3B allows the selection of a parental combinations for a subsequent crossing 124, showing heterotic hybrid seeds having their seed weight significantly higher than its parents (for example, in Table 3A, the cross between 106 as female and 102 as male, and in Table 3B, the cross between 3097 as female and 3028 as male), as well as cases with heterotic smaller hybrid seeds than both parent seeds, such as e.g., in Table 3A, the parental combination of 108 as female parent and 109 as male resulting in hybrid seeds weights which are significantly lower than both parents. Moreover, hybrid seeds derived from the reciprocal cross of 108 and 109 (109 as female and 108 as male) are significant larger than the non-hybridized seeds of the male parent (see Table 3A). These results show that hybrid seeds size may be determined by the specific combination of the parents, a factor the inventors have found out that can be used for the disclosed breeding and production methods. Theses tables provide initial information which is being extended enhanced in the breeding plan.

Tables 3A and 3B: An example for the crossing stage (124) in the breeding of parental lines for hybrid production in cotton (Gossypium spp.)—Table 3A, and in pea (Pisum sativum)—Table 3B. The parental lines (each line is described by its number and also by species name and ploidy level, 9 parental lines in Table 3A, 13 parental lines in Table 3B) were used to produce crossing matrix where each of the lines function as both female (x-axis) and male (y-axis). Each square contains the resulted for a unique parental combination. At least twenty seeds were measured for each combination and a mean seed weight and Standard Deviation (SD, a; in parenthesis) were calculated. The results of the self-pollinations are presented in the diagonal square (grey background) in different type faces: Regular font indicates that the hybrid seed weight is intermediate between the parents; Bold font indicates that the hybrid seed weight is significantly different from one parent; Italics font indicates that the hybrid seed weight is significantly lower than both parents; Italics bold font indicates that the hybrid seed weight is significantly higher than both parents.

| | Male | | | | | | |
|---|---|---|---|---|---|---|---|
| Female | 3010 | 3021 | 3028 | 3031 | 3042 | 3050 | 3066 |
| 3010 | 0.326(0.036) | | | | | | |
| 3021 | | 0.288(0.052) | | | | | |
| 3028 | | | 0.238(0.035) | | | | |
| 3031 | | 0.071(0.020) | | 0.065(0.009) | | | |
| 3042 | | | | | 0.168(0.031) | | |
| 3050 | | | | | | 0.170(0.020) | |
| 3066 | | | | | | 0.421(0.023) | 0.404(0.054) |
| 3078 | | | | | | | |
| 3097 | | | 0.405(0.067) | | | | |
| 3148 | | | | | | | |
| 4028 | | | | | | | |
| 4045 | | | | | | | |
| 4123 | | | | | | | |
| | | Male | | | | | |
| | Female | 3078 | 3097 | 3148 | 4028 | 4045 | 4123 |
| | 3010 | | | | 0.306(0.033) | | |
| | 3021 | | | | | | |
| | 3028 | | | | | | |
| | 3031 | | | | | | |
| | 3042 | | | | | | |
| | 3050 | | | | | | |
| | 3066 | | | | | | 0.351(0.013) |
| | 3078 | 0.293(0.027) | | | 0.416(0.029) | | |
| | 3097 | | 0.336(0.052) | | | | 0.285(0.052) |
| | 3148 | | | 0.468(0.034) | 0.466(0.032) | 0.482(0.031) | |
| | 4028 | | | | 0.238(0.035) | | |
| | 4045 | | | | | 0.218(0.084) | |
| | 4123 | | | | | | 0.058(0.011) |

TABLE 3C
variety of seeds average size in hybrids
with respect to parent varieties.

| | | hybrid seeds average size | | | |
|---|---|---|---|---|---|
| | | | | | differ |
| | # of | lower than | | | from 1 |
| crop | crosses | both | intermediate | higher than both | parent |
| Cotton | 13 | 4 | 4 | 1 | 4 |
| Cowpea | 16 | | 8 | | 8 |
| Pea | 10 | | 1 | 2 | 7 |

It is noted that for the studied and bred parent varieties, hybrid seeds exhibit a range of relations between their seed characteristics and the seed characteristics of their respective parent varieties, which may be made more pronounced by selection and breeding methods disclosed herein, to yield hybrids with heterotic seed traits with respect to their parent varieties.

In certain embodiments, a pair of parents showing the highest distance in the DC values of the hybrid seeds and the parental self-seeds may be selected 126 for further breeding or for hybrid production 110.

It is noted that pair selection 126 may be used to change the starting parent pairs in order to achieve more favorable starting points with respect to the distribution parameters or with respect to the seed characteristic selected for separation.

The selected pair may contain the highest distance value, lower or upper compare to all measured crossing pairs. In certain embodiments, the distance between the seed lots increases differentiation (separation 140 and/or 145) efficiency, while in other embodiments, parent varieties which are close to each other with respect to the heterotic trait may allow better separation of the heterotic hybrid seeds from the parents'seeds. In case the distance value is lower than the separation machine limitation or even smaller than achieved before, crossing and selection 124-126 or generally breeding 120 may be reiterated until the required traits and seed characteristic(s) values are reached.

Table 4 provides a non-limiting list of prospective crops to be considered for application of certain embodiments. These crops exhibit a potential for seed differentiation as disclosed herein and were further selected according to their seed type (and ability to reach the disclosed separation) and commercial aspects to be appropriate candidates for application of the disclosed methods. It is noted that the crop species belong to a wide range of families with widely spread seed characteristics and pollination mechanisms. Initial experiments have validated the potential of certain embodiments in peas, cowpeas and cotton, and ongoing experiments are carried out for validation in other crops. In particular, crops with non-endospermic or perispermic seed types may be appropriate candidates for application of the disclosed methods.

TABLE 4
Exemplary list of candidate crops.

| Species | Seed type | Family name |
|---|---|---|
| Garlic, Onions | Endospermic | Amaryllidaceae |
| Sesame | Endospermic | Pedaliaceae |
| Carrots | Endospermic | Apiaceae |
| Sunflower seed | Non-endospermic | Asteraceae |
| Cabbage, Mustard seed, Rapeseed | Non-endospermic | Brassicaceae |
| Sugar beet | Perispermic | Chenopodiaceae |
| Cucumber, Melons, Pumpkins, | Non-endospermic | Cucurbitaceae |
| Squash, Watermelons | | |
| Castor oil seed | Endospermic | Euphorbiaceae |
| Beans, Chickpeas, Cloves, | Non-endospermic | Faboideae |
| Groundnuts, Lentils, Lupins, | | |
| Pea, Soybeans, Vetches, Cowpea, | | |
| broad bean | | |
| Cotton | Non-endospermic | Malvaceae |
| Eggplants, Pepper, Potato, Tomato | Non-endospermic | Solanaceae |

Table 5 illustrates the stability of seed weight as the seed characteristic, over multiple parents in cotton, peas and cowpeas. The stability is important to enable a differentiation and consequently a separation between the variability of hybrid seeds from a given pair of parent types and the variability of hybrid seed from different types of parents. One of the criteria for selecting the specific parents may be the stability of the seed characteristic of their hybrids.

TABLE 5
Illustration of hybrid seed weight stability
in cotton, pea and cowpea.

| | Parent | # of | | | |
|---|---|---|---|---|---|
| Crop | name | replicates | AV | SD | CV (%) |
| Cotton | P1 | 20 | 72.45 | 6.76 | 9.33 |
| Cotton | P2 | 20 | 65.75 | 5.29 | 8.05 |
| Cotton | P3 | 20 | 72.55 | 7.37 | 10.16 |
| Cotton | P4 | 20 | 90.85 | 20.15 | 22.18 |
| Cotton | P5 | 20 | 66.76 | 4.11 | 6.16 |
| Cotton | P6 | 20 | 114.40 | 13.43 | 11.74 |
| Cotton | P7 | 20 | 145.45 | 9.63 | 6.62 |
| Cotton | P8 | 20 | 155.05 | 15.24 | 9.83 |
| Cotton | P9 | 20 | 112.80 | 14.67 | 13.01 |
| Cowpea | P1 | 21 | 255.48 | 49.88 | 0.20 |
| Cowpea | P2 | 21 | 102.33 | 14.44 | 0.14 |
| Cowpea | P3 | 21 | 119.14 | 12.49 | 0.10 |
| Cowpea | P4 | 11 | 71.09 | 17.70 | 0.25 |
| Cowpea | P5 | 10 | 315.50 | 84.80 | 0.27 |
| Cowpea | P6 | 21 | 102.24 | 32.86 | 0.32 |
| Cowpea | P7 | 21 | 190.29 | 26.51 | 0.14 |
| Cowpea | P8 | 21 | 320.05 | 69.52 | 0.22 |
| Cowpea | P9 | 21 | 365.81 | 52.96 | 0.14 |
| Cowpea | P10 | 10 | 87.70 | 15.00 | 0.17 |
| Cowpea | P11 | 10 | 112.60 | 17.98 | 0.16 |
| Cowpea | P12 | 11 | 122.55 | 25.29 | 0.21 |
| Cowpea | P13 | 11 | 230.73 | 15.46 | 0.07 |
| Cowpea | P14 | 11 | 76.55 | 8.43 | 0.11 |
| Cowpea | P15 | 21 | 66.10 | 9.42 | 0.14 |
| Cowpea | P16 | 11 | 305.64 | 26.72 | 0.09 |
| Cowpea | P17 | 21 | 218.71 | 66.66 | 0.30 |
| Cowpea | P18 | 21 | 109.76 | 26.91 | 0.25 |
| Pea | P1 | 21 | 301.19 | 27.17 | 0.09 |
| Pea | P2 | 21 | 331.90 | 36.91 | 0.11 |
| Pea | P3 | 21 | 67.57 | 9.80 | 0.15 |
| Pea | P4 | 21 | 295.48 | 52.43 | 0.18 |
| Pea | P5 | 21 | 59.76 | 11.70 | 0.20 |
| Pea | P6 | 21 | 203.33 | 36.87 | 0.18 |
| Pea | P7 | 21 | 239.19 | 35.18 | 0.15 |
| Pea | P8 | 21 | 336.05 | 52.20 | 0.16 |
| Pea | P9 | 21 | 396.14 | 65.85 | 0.17 |
| Pea | P10 | 21 | 469.14 | 34.77 | 0.07 |
| Pea | P11 | 21 | 218.33 | 84.08 | 0.39 |
| Pea | P12 | 21 | 167.43 | 31.67 | 0.19 |
| Pea | P13 | 21 | 170.00 | 20.15 | 0.12 |

Hybrid production 110 may be based on natural pollination using biotic and/or abiotic vectors to produce hybrid seeds. The selected pairs of the parental lines may be sown in open field for pollination. In case of insect pollination, the field may be separated from other fields with compatible crops to avoid cross contamination. In case of abiotic pollination, the field may be separated according to parameters related to the pollination agent. For example, in the case of wind pollination, the different pairs of parental lines can be separated with respect to the wind direction.

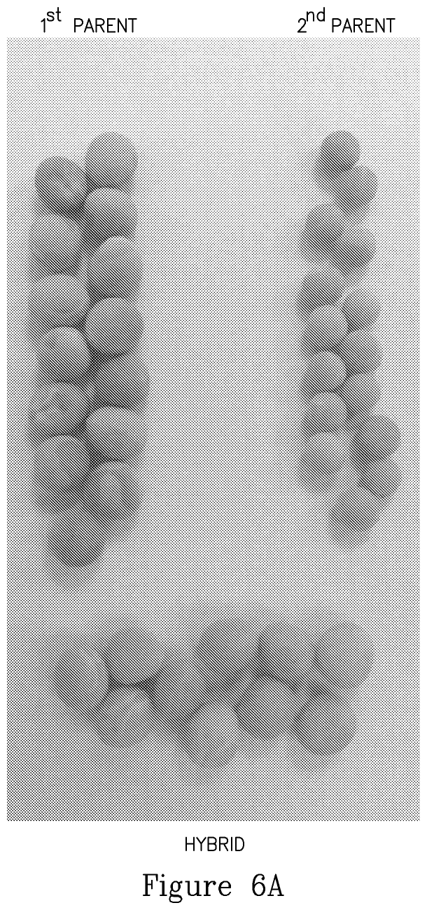

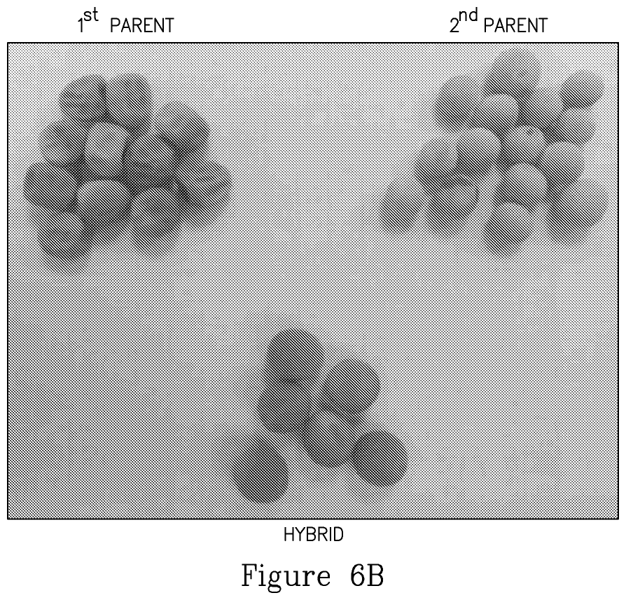

FIGS. 6A and 6B provide examples for separation of hybrid seeds of cowpeas and peas, respectively, such as seed color providing separation between the first (female—pollen receiving) parent and seed size providing separation between the second (male—pollen donating) parent, according to some embodiments of the invention. In both cases, seed size provides a non-limiting example for a heterotic trait, in these cases the hybrid seeds are larger than the seeds of both parent varieties. Additional traits that may be used for supporting hybrid separation are seed color (green vs. yellow), e.g., for separating the female parent seeds from the hybrid seeds illustrated in FIG. 6A; and surface texture (wrinkled versus smooth), e.g., for separating the female parent seeds from the hybrid seeds illustrated in FIG. 6B.

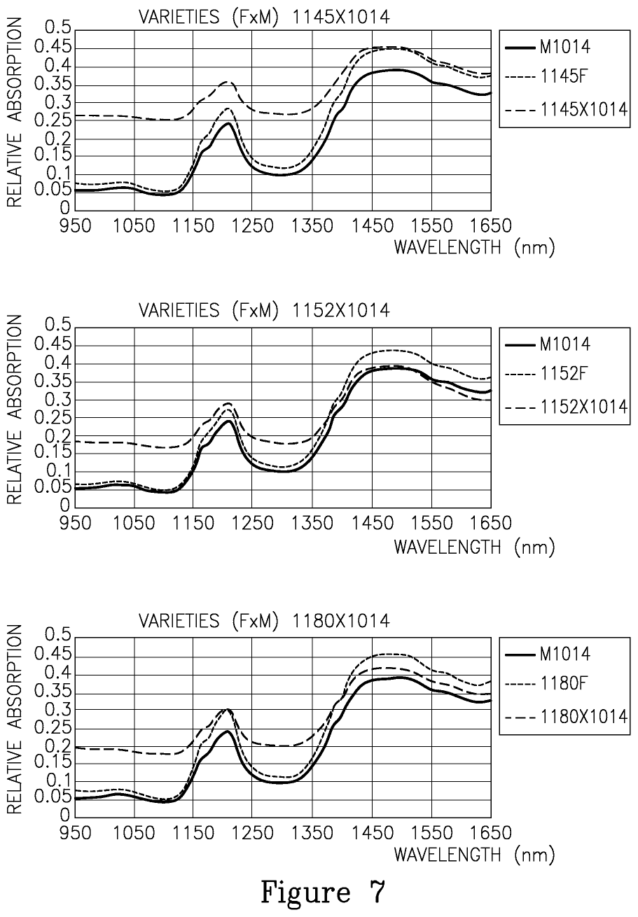

FIG. 7 provides examples for separation of hybrid seeds of sesame from the respective parent seeds using near infrared (NIR) spectroscopy, according to some embodiments of the invention. Separation between the male common parent, three female parents and the respective three hybrids which exhibit heterotic effect in multiple wavelengths, e.g., 1100 nm, 1300 nm, ranges in between, etc. NIR spectroscopy absorption spectrum therefore provides a non-limiting example for a heterotic trait of the hybrid seeds (in these cases the hybrid seeds absorption rate is greater than the seeds of both parent varieties).

In the above description, an embodiment is an example or implementation of the invention. The various appearances of “one embodiment”, “an embodiment”, “certain embodiments” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Certain embodiments of the invention may include features from different embodiments disclosed above, and certain embodiments may incorporate elements from other embodiments disclosed above. The disclosure of elements of the invention in the context of a specific embodiment is not to be taken as limiting their used in the specific embodiment alone.

Furthermore, it is to be understood that the invention can be carried out or practiced in various ways and that the invention can be implemented in certain embodiments other than the ones outlined in the description above.

The invention is not limited to those diagrams or to the corresponding descriptions. For example, flow need not move through each illustrated box or state, or in exactly the same order as illustrated and described.

Meanings of technical and scientific terms used herein are to be commonly understood as by one of ordinary skill in the art to which the invention belongs, unless otherwise defined.

While the invention has been described with respect to a limited number of embodiments, these should not be construed as limitations on the scope of the invention, but rather as exemplifications of some of the preferred embodiments. Other possible variations, modifications, and applications are also within the scope of the invention. Accordingly, the scope of the invention should not be limited by what has thus far been described, but by the appended claims and their legal equivalents.

1. A method of obtaining specified hybrid seeds, the method comprising:
growing two varieties of a field crop, wherein the two varieties:
the varieties are at least partially cross-pollinated in the field,
are fertile with respect to both male and female functions,
are selected to yield at least one specified hybrid, and
are distinguishable with respect to at least one seed characteristic, collecting seeds from the grown field crop, and
separating, from the collected seeds, at least one fraction of hybrid seeds of the at least one specified hybrid, wherein the at least one separated fraction is distinct and separable, with respect to the at least one seed characteristic, from seed fractions of the non-hybridized two varieties.

2. The method of claim 1, wherein the distinction comprises a separability of the at least one fraction of hybrid seeds from the fractions of the non-hybridized two varieties.

3. The method of claim 2, wherein a mean of the at least one seed characteristic of the at least one fraction of hybrid seeds is either larger or smaller than both means of the at least one seed characteristic of the fractions of the non-hybridized two varieties.

4. The method of claim 3, wherein the mean of the at least one seed characteristic of the at least one fraction of hybrid seeds is separated from the mean of the at least one seed characteristic of the fraction of an adjacent non-hybridized variety by at least a sum of standard deviations thereof.

5. The method of claim 1, further comprising breeding at least one of the two varieties to yield the distinction and separability.

6. The method of claim 1, wherein the two varieties are selected to yield at least two specified hybrids, a first hybrid resulting from pollination of a first variety by a second variety and a second hybrid resulting from pollination of the second variety by the first variety.

7. The method of claim 1, further comprising sowing the two varieties together as one mixture.

8. The method of claim 1, wherein the at least one seed characteristic relates to a heterotic trait.

9. The method of claim 1, wherein the mean values and the standard deviations are statistical measures of empirical distributions of the respective seed fractions.

10. The method of claim 1, wherein the field crop has a non-endospermic or a perispermic seed type.

11. The method of claim 10, wherein the field crop is one of: sunflower, cabbage, mustard, rapeseed, sugar beet, cucumber, melon, pumpkin, squash, watermelon, bean, chickpea, cowpea, broad bean, clove, groundnut, lentil, lupine, pea, soybean, vetch, cotton, eggplant, pepper, potato and tomato.

12. The method of claim 11, wherein the field crop is cowpea or cotton.

13. Hybrid seeds produced by the method of claim 1.

14. Hybrid seeds of two varieties of a field crop which are at least partially bi-directionally cross-pollinated in the field, wherein the two varieties:
are fertile with respect to both male and female functions,
are selected to yield at least one specified hybrid, and
are distinguishable with respect to at least one seed characteristic,
wherein, in collected seeds of the field crop, at least one fraction of hybrid seeds of the at least one specified hybrid is distinct and separable, with respect to the at least one seed characteristic, from seed fractions of the non-hybridized two varieties.

15. The hybrid seeds of claim 14, wherein a mean of the at least one seed characteristic of the at least one fraction of hybrid seeds is either larger or smaller than both means of the at least one seed characteristic of the fractions of the non-hybridized two varieties.

16. The hybrid seeds of claim 15, wherein the mean of the at least one seed characteristic of the at least one fraction of hybrid seeds is separated from the mean of the at least one seed characteristic of the fraction of an adjacent non-hybridized variety by at least a sum of standard deviations thereof.

17. The hybrid seeds of claim 14, wherein the field crop has a non-endospermic or a perispermic seed type.

18. The hybrid seeds of claim 14, wherein the field crop is one of: sunflower, cabbage, mustard, rapeseed, sugar beet, cucumber, melon, pumpkin, squash, watermelon, bean, chickpea, cowpea, broad bean, clove, groundnut, lentil, lupine, pea, soybean, vetch, cotton, eggplant, pepper, potato and tomato.

19. The hybrid seeds of claim 18, wherein the field crop is cowpea or cotton.
